(19) United States
(12) Patent Application Publication
(10) Pub. No.: US 2002/0137086 A1
(43) Pub. Date: Sep. 26, 2002

(54) Method for the development of gene panels for diagnostic and therapeutic purposes based on the expression and methylation status of the genes

(76) Inventors: Alexander Olek, Berlin (DE); Kurt Berlin, Stahnsdorf (DE)

Correspondence Address: KRIEGSMAN & KRIEGSMAN, 665 Franklin Street, Framingham, MA, 01702, US

(21) Appl. No.: 10/087,466

(22) Filed: Mar. 1, 2002

Related U.S. Application Data
(60) Provisional application No. 60/272,549, filed on Mar. 1, 2001.

(51) Int. Cl. C12Q 1/68; G06F 19/00; G01N 33/48; G01N 33/50

(52) U.S. Cl. 435/6; 702/20

(57) ABSTRACT

A method for the development of gene panels for diagnostic and therapeutic purposes comprising the steps of: (a) isolating at least one biological sample from each of at least two groups of biological material containing mRNA and/or proteins; (b) analyzing the expression level of at least one gene in the at least one biological sample; (c) selecting the gene(s) exhibiting a different expression level between the at least two groups of biological material, whereby a first knowledge base is generated; (d) analyzing the level of cytosine methylation in the methylation relevant regions of at least one gene of at least one of the biological samples of step (a), wherein the gene is selected on the basis of the first knowledge base; (e) selecting the gene(s) exhibiting a different level of cytosine methylation between the at least two groups of biological material, whereby a second knowledge base is generated; and (f) adding selected genes from the second knowledge base to a gene panel.

### FIELD OF THE INVENTION

The present invention concerns a method for the development of gene panels for diagnostic and therapeutic purposes. The invention further concerns gene panels developed using the method of the present invention and their uses.

### BACKGROUND OF THE INVENTION

The levels of observation that have been well studied by the methodological developments of recent years in molecular biology include the gene itself, the translation of genes in RNA, and the resulting proteins. When, during the course of the development of an individual, a gene is switched on, and how the activation and inhibition of certain genes in certain cells and tissues is controlled, can be correlated with a high degree of probability with the extent and the character of the methylation of the gene or the genome. In this regard, it is reasonable to assume that pathogenic conditions are expressed in a modified methylation pattern of individual genes or of the genome.

### STATE OF THE ART

1. State of the Art of Molecular Analysis of Cell Phenotypes

The study of gene expression can be at the RNA level or at the protein level. Both levels in principle reflect important phenotypic parameters. Protein assays using two-dimensional gels (McFarrel method) have been known for approximately 15 years. Using these assays, it is possible to elaborate the analysis of the chromatographic positions of several thousand proteins. Very early on, such electropherograms were already processed or evaluated with data processing means. In principle, the validity of the method is high, however, it is inferior to the modern methods of gene expression based on RNA analysis in two regards.

The global analysis of cellular proteins has recently been termed proteomics and is a key area of research that is developing in the post-genome era. Proteomics uses a combination of sophisticated techniques including two-dimensional (2D) gel electrophoresis, image analysis, mass spectrometry, amino acid sequencing, and bio-informatics to resolve comprehensively, to quantify, and to characterize proteins (for reviews, see Chambers G et al. “Proteomics: a new approach to the study of disease” J Pathol November 2000; 192(3):280-8; Banks R E et al. “Proteomics: new perspectives, new biomedical opportunities” Lancet Nov. 18, 2000;356(9243):1749-56).

Proteomics is further said to contribute greatly to the understanding of gene function in the post-genomic era. Proteomics can be divided into three main areas: (1) protein micro-characterization for large-scale identification of proteins and their post-translational modifications; (2) ‘differential display’ proteomics for comparison of protein levels with potential application in a wide range of diseases; and (3) studies of protein-protein interactions using techniques such as mass spectrometry or the yeast two-hybrid system. Because it is often difficult to predict the function of a protein based on homology to other proteins or even their three-dimensional structure, determination of the components of a protein complex or of a cellular structure is central in functional analysis (Pandey A et al. “Proteomics to study genes and genomes.” Nature Jun. 15, 2000;405(6788):837-46).

Due to the complexity of higher eukaryotic cells, single-step characterization of a proteome is likely to be difficult to achieve. Jung et al. (“Proteomics meets cell biology: the establishment of subcellular proteomes” Electrophoresis October 2000;21(16):3369-77) describe, that advantage can be taken of the macromolecular architecture of a cell, e.g., subcellular compartments, organelles, macromolecular structures and multi-protein complexes, to establish subcellular proteomes.

Despite the recent developments in the art, the detection of proteins that are of regulatory importance, from small quantities of cells still fails because of the fact that the sensitivity of the methods used is much too low. Indeed, in contrast to nucleic acids, proteins cannot be amplified. In addition, the method is very complex, not amenable to automation, and very expensive. In contrast, RNA analysis presents considerable advantages, and due to of the use of PCR it is more sensitive. Above all, each RNA species recognized to be important can be identified immediately by its sequence.

Overexpression or underexpression of individual RNAs with a known sequence can usually be easily detected; however, in connection with the applications discussed here, they are only valid in exceptional cases.

The method of “differential displays” at best allows a semiquantitative study of expression. Expression products amplified by PCR are separated by gel electrophoresis. The validity is limited as a result of the resolution of the gel electrophoresis. In addition, the method is insufficiently sensitive and robust for use in routine diagnosis (Liang, P. and Pardee, A. B., Science 257, 967-971).

Genes with high overexpression or underexpression are frequently identified by subtractive techniques. Here, cDNA clones of a cell or tissue species to be examined are plated. Against the clones, cDNA is hybridized as comparison material. Expression patterns cannot be reliably prepared using this technique.

Sturtevant et al. (“Applications of differential-display reverse transcription-PCR to molecular pathogenesis and medical mycology. Clin Microbiol Rev July 2000;13(3):408-27) describe the characterisation of host-fungus interactions by changes in gene expression in both host and pathogen. Differential-display reverse transcription PCR (DDRT-PCR) is a PCR-based method that allows extensive analysis of gene expression among several cell populations. DDRT-PCR has been used to address biological questions in mammalian systems, including cell differentiation, cell activation, cell stress, and identification of drug targets. In microbial pathogenesis and plant pathogenesis, DDRT-PCR has allowed the identification of virulence factors, genes involved in cell death, and signaling genes. Further, To (“Identification of differential gene expression by high throughput analysis” Comb Chem High Throughput Screen June 2000;3(3):235-41) describes the high throughput analysis of differential gene expression as a powerful tool that can be applied to many areas in molecular cell biology, including differentiation, development, physiology, and pharmacology. In recent years, a variety of techniques have been developed to analyze differential gene expression, including comparative expressed sequence tag sequencing, differential display, representational difference analysis, cDNA or oligonucleotide arrays, and serial analysis of gene expression. Similar strategies are described by Oetting (“Gene expression analysis. Pigment Cell Res February 2000;13(1):21-7) and Watson (“Differential cDNA screening strategies to identify novel stage-specific proteins in the developing mammalian brain. Dev Neurosci 1993;15(2):77-86).

One activity of the American “Human Genome Project” is the systematic sequencing of expressed genes. The data obtained from this can be used to build expression chips, which allow the study of practically all expressed sequences of a cell or tissue type in a single experiment.

2. State of the Art in the Analysis of Cancer Diseases

Mutations in genes can trigger cancer diseases, that is, cell transformation. The causes of these mutations can be exogenous influences, or events in the cell. In a few exceptional cases, an individual mutation, which frequently affects larger regions of the genome (translocations, deletions), results in the degeneration of the cell; but in most cases a chain of mutations on different genes is involved, and it is only their combined effect that results in the malignant disease. These changes at the DNA level are also reflected on the RNA and protein levels. In this context, it is highly probable that a multiplication occurs, because it is certain that in many cases the quantity and type of one RNA influences the extent of the synthesis of several other RNA species. This leads to a change in the synthesis rates of the corresponding proteins, which, in turn, can result in the deregulation of metabolism, and thus initiate the mechanism of regulation and counter regulation. The result is a gene expression pattern of the cells in question, that has been modified in a very specific (but largely nondeterminable) manner,the specificity is for a certain carcinoma, for the stage of the carcinoma, and the degree of malignancy of the carcinoma. So far, such phenomena have been outside the realm of study of natural sciences. Indeed, it has been impossible to examine the gene expression or the metabolism of a cell in its totality. Chip technology for the first time provided such a possibility (Schena, M. et al., Science 270, 467-470).

If one wishes to solve the diagnostic problem of early diagnosis of tumors on the molecular level, then one is confronted, today, with an insurmountable difficulty. With very few exceptions, for most tumors, the knowledge of the molecular events, that is, the different mutations, is only fragmentary. Researchers do not know what to look for in medical examination material. This means it is absolutely impossible to apply the remarkable sensitivity and specificity of the polymerase chain reaction. Examples are certain intestinal tumors, Ewing's sarcoma, and certain forms of leukemia, which are in fact each defined by a single, precisely described mutation. In those cases, it is possible to identify the degenerated cell among millions of normal cells. However, even within these apparently unambiguously defined tumor groups, there are such differences in the behavior that the conclusion must be drawn that additional unknown genetic parameters (such as, for example, the genetic background of the individual) play an important role. Immunological tumor markers are helpful auxiliary parameters, but they continue to make only a modest contribution, in addition to the other conventional diagnostic parameters. However, they can be used for the purpose of preselecting suspect cells.

Histology plays an important and indispensable role in the identification of degenerated tissues, but not precisely in early diagnosis.

Thus, because most tumors are not sufficiently characterized for diagnostic purposes on the molecular level, as a rule, no possibilities exist to proceed to a subdivision into stages or even a subdivision by degrees of risk. Such a subdivision, however, is an absolute prerequisite for an improved selection of treatments and, above all, for the development of effective new drugs and of gene therapy.

3. State of the Art in Research on the Number, Type and Properties of the Possible Stable States of Cells of Higher Organisms

In recent times, there has been an increase in the number of indications that complex regulatory systems (an excellent example of which is cell regulation), when left alone, can exist in only a limited number of stable states, above a critical minimum complexity and below a critical maximum connectivity (of the average number of the components, with which any given component is connected) (Kauffman, S. A., Origins of Order, Oxford University Press, 1993). In this context, the word state should be understood as the concept of selection for the general phenomenon. In connection with cells as biological regulatory systems, one can also talk of differentiation state or cell type. Although no such connection has been demonstrated—and even a mere limitation of the possible states for biological systems has not been demonstrated—the practical implications would be of very great importance: if, regarding the constant information content of the cells of an organism (de facto, such constancy essentially exists within one species), there were only a limited number of stable states, then it would be likely that degenerated cells could also be in only one of these states or in a transition between the possible states. At this time, it is not possible to define these states on a molecular basis. It is hardly possible to achieve a correlation between the individual states and the behavior of the cells according to the state of the art. However, such an analysis could make decisive contributions to the diagnosis and prognosis of diseases. It is even possible that a correlation could be established between the possible states of diseased cells and the best suited therapy. Furthermore, it is probable that such a method could also have a decisive influence in the selection of the time of treatment. For example, if one were to discover that the cells of a tumor are in a transition between possible states, one could assume that such a population of cells would be more likely to yield to the selection pressure resulting from the treatment, and thus could escape more easily. A cell population in such a scenario, within such transitional states, would have a considerably increased flexibility, and it would be easily forced into a possible stable state, in which the selection pressure would be eliminated, and the treatment would thus be without effect.

A method which could classify cells and cell groups according to states would then also contribute to recognizing, understanding and possibly solving such problems. However, according to the state of the art, it is not possible to determine whether only a limited number of states of cells exists. It follows that it is not possible to differentiate groups of cells according to an abstract criterion concerning their states, and to predict these states with a certain behavior of the cells.

4. Hereditary Diseases

Today, the genetic map of the human genome comprises 2500 so-called microsatellites. These instruments are used to locate a multitude of genes, usually genes whose defect causes a genetic disease, per linkage analysis, and then to identify them. Common genetic diseases caused by a single defective gene are thus elucidated, from the point of view of the geneticist's principle, polygenic diseases should also be understood in this manner. Many polygenic diseases are very common, so common that they are included among the so-called wide-spread diseases. Asthma and diabetes are examples. Many carcinoma types are also included. The use of the above described strategy of linkage analysis also produced enormous initial successes. In many instances, numerous causal genes of important polygenic diseases such as diabetes, schizophrenia, atherosclerosis and obesity have been found. Besides the availability of the molecular biology laboratory techniques proper, the availability of a relatively large number of patients and relatives affected by each disease is a crucial prerequisite for genetic elucidation. In the past two years it has become apparent that the number of several hundred patients that were originally used for the linkage analysis of polygenic diseases very likely is too low by one order of magnitude. This applies, in any case, to cases where the entire spectrum of the causal gene is to be elucidated. Because the level of manual work required for such a linkage analysis is extraordinarily high, only very slow progress can be expected in the analysis of polygenic diseases. Alternative strategies are sought because it is precisely these diseases that are of enormous social and economic importance.

5. State of the Art in Methylation Analysis

The modification of the genomic base cytosine to 5′-methylcytosine represents the epigenetic parameter which to date is the most important one and has been best examined. Nevertheless, methods exist today to determine comprehensive genotypes of cells and individuals, but no comparable methods exist to date to generate and evaluate epigenotypic information on a large scale.

In principle, there are three methods that differ in principle for determining the 5-methyl state of a cytosine in the sequence context.

The first method is based in principle on the use of restriction endonucleases (RE), which are methylation-sensitive”. REs are characterized in that they produce a cut in the DNA at a certain DNA sequence which is usually 4-8 bases long. The position of such cuts can be detected by gel electrophoresis, transfer to a membrane and hybridization. Methylation-sensitive means that certain bases within the recognition sequence must be unmethylated for the step to occur. The band pattern after a restriction cut and gel electrophoresis thus changes depending on the methylation pattern of the DNA. However, most CpG that can be methylated are outside of the recognition sequences of REs, and thus cannot be examined.

The sensitivity of this method is extremely low (Bird, A. P., Southern, E. M., J. Mol. Biol. 118, 27-47). A variant combines PCR with this method; an amplification by two primers located on both sides of the recognition sequence occurs after a cut only if the recognition sequence is in the methylated form. In this case, the sensitivity theoretically increases to a single molecule of the target sequence; however, only individual positions can be examined, at great cost (Shemer, R. et al., PNAS 93, 6371-6376).

The second variant is based on the partial chemical cleavage of whole DNA, using the model of a Maxam-Gilbert sequencing reaction, ligation of adaptors to the ends thus generated, amplification with generic primers, and separation by gel electrophoresis. Using this method, defined regions having a size of less than thousands of base pairs can be examined. However, the method is so complicated and unreliable that it is practically no longer used (Ward, C, et al., J. Biol. Chem. 265, 3030-3033).

A new method for the examination of DNA to determine the presence of 5-methylcytosine is based on the specific reaction of bisulfite with cytosine. The latter is converted under appropriate conditions into uracil, which, as far as base pairing is concerned, is equivalent to thymidine, and which also corresponds to another base. 5-Methylcytosine is not modified. As a result, the original DNA is converted in such a manner that methylcytosine, which originally could not be distinguished from cytosine by its hybridization behavior, now can be detected by “normal” molecular biological techniques. All of these techniques are based on base pairing, which can now be completely exploited. The state of the art, as far as sensitivity is concerned, is defined by a method which includes the DNA to be examined in an agarose matrix, intended to prevent the diffusion and renaturing of the DNA (bisulfite reacts only with singlestranded DNA) and to replace all precipitation and purification steps by rapid dialysis (Olek, A., et al., Nucl. Acids. Res. 24, 5064-5066). Using this method, individual cells can be examined, which illustrates the potential of the method. However, so far only individual regions up to approximately 3000 base pairs in length have been examined, and an overall examination of cells to identify thousands of possible methylation events is not possible. However, this method is not capable of reliably analyzing minute fragments from small sample quantities. In spite of protection against diffusion, such samples are lost through the matrix.

6. State of the Art with Respect to Methylation and the Diagnosis of Human Diseases

In the past, modification of the methylation pattern was analyzed in order to study and understand the genetic mechanisms which are involved in the outbreak or the progression of a disease. All this research was done in a piece-by-piece fashion by studying only one gene/chromosomal region at a time and no diagnosis/therapeutic treatment regimen was based on the methylation pattern modifications. In fact, the type of disease associated with the modification of the methylation pattern was known before methylation analysis was performed. Therefore, the following publications only indicate the widespread connection between modifications of the methylation patterns and human diseases. Modifications can include both hyper- or hypomethylation of selected sites of the DNA.

Disease associated with a modification of the methylation patterns are, for example:

Leukemia (Aoki E et al. “Methylation status of the p15INK4B gene in hematopoietic progenitors and peripheral blood cells in myelodysplastic syndromes” Leukemia April 2000;14(4):586-93; Nosaka K et al. “Increasing methylation of the CDKN2A gene is associated with the progression of adult T-cell leukemia” Cancer Res Feb. 15, 2000;60(4):1043-8; Asimakopoulos FA et al. “ABL1 methylation is a distinct molecular event associated with clonal evolution of chronic myeloid leukemia” Blood Oct 1, 1999;94(7):2452-60; Fajkusova L. et al. “Detailed Mapping of Methylcytosine Positions at the CpG Island Surrounding the Pa Promoter at the bcr-abl Locus in CML Patients and in Two Cell Lines, K562 and BV173” Blood Cells Mol Dis June 2000;26(3):193-204; Litz C E et al. “Methylation status of the major breakpoint cluster region in Philadelphia chromosome negative leukemias” Leukemia January 1992;6(1):35-41)

Head and neck cancer (Sanchez-Cespedes M et al. “Gene promoter hypermethylation in tumors and serum of head and neck cancer patients” Cancer Res Feb. 15, 2000;60(4):892-5)

Hodgkin's disease (Garcia J F et al. “Loss of p16 protein expression associated with methylation of the p16INK4A gene is a frequent finding in Hodgkin's disease” Lab Invest December 1999;79(12):1453-9)

Gastric cancer (Yanagisawa Y et al. “Methylation of the hMLH1 promoter in familial gastric cancer with microsatellite instability” Int J Cancer Jan. 1, 2000;85(1):50-3)

Prostate cancer (Rennie P S et al. “Epigenetic mechanisms for progression of prostate cancer” Cancer Metastasis Rev 1998-99;17(4):401-9)

Renal cancer (Clifford S C et al. “Inactivation of the von Hippel-Lindau (VHL) tumor suppressor gene and allelic losses at chromosome arm 3p in primary renal cell carcinoma: evidence for a VHL-independent pathway in clear cell renal tumourigenesis” Genes Chromosomes Cancer July 1998;22(3):200-9)

Bladder cancer (Sardi I et al. “Molecular genetic alterations of c-myc oncogene in superficial and locally advanced bladder cancer” Eur Urol 1998;33(4):424-30)

Breast cancer (Mancini D N et al. “CpG methylation within the 5′ regulatory region of the BRCA1 gene is tumor specific and includes a putative CREB binding site” Oncogene Mar. 5, 1998;16(9):1161-9; Zrihan-Licht S et al. “DNA methylation status of the MUC1 gene coding for a breast-cancer-associated protein” Int J Cancer Jul. 28, 1995;62(3):245-51; Kass D H et al. “Examination of DNA methylation of chromosomal hot spots associated with breast cancer” Anticancer Res September-October 1993;13(5A):1245-51)

Burkitt's lymphoma (Tao Q et al. “Epstein-Barr virus (EBV) in endemic Burkitt's lymphoma: molecular analysis of primary tumor tissue” Blood Feb. 15, 1998;91(4):1373-81)

Wilms tumor (Kleymenova E V et al. “Identification of a tumor-specific methylation site in the Wilms tumor suppressor gene” Oncogene Feb. 12, 1998;16(6):713-20)

Prader-Willi/Angelman syndrome (Zeschnigh et al. “Imprinted segments in the human genome: different DNA methylation patterns in the Prader-Willi/Angelman syndrome region as determined by the genomic sequencing method” Human Mol. Genetics (1997) (6)3 pp 387-395; Fang P et al. “The spectrum of mutations in UBE3A causing Angelman syndrome” Hum Mol Genet January 1999;8(1):129-35)

ICF syndrome (Tuck-Muller et al. “CMDNA hypomethylation and unusual chromosome instability in cell lines from ICF syndrome patients” Cytogenet Cell Genet 2000;89(1-2):121-8)

Dermatofibroma (Chen T C et al. “Dermatofibroma is a clonal proliferative disease” J Cutan Pathol January 2000; 27(1):36-9)

Hypertension (Lee S D et al. “Monoclonal endothelial cell proliferation is present in primary but not secondary pulmonary hypertension” J Clin Invest Mar. 1, 1998;101(5):927-34)

Pediatric Neurobiology (Campos-Castello J et al. “The phenomenon of genomic “imprinting” and its implications in clinical neuropediatrics” Rev Neurol Jan. 1-15, 1999;28(1):69-73)

Autism (Klauck S M et al. “Molecular genetic analysis of the FMR-1 gene in a large collection of autistic patients” Hum Genet August 1997;100(2):224-9)

Ulcerative colitis (Gloria L et al. “DNA hypomethylation and proliferative activity are increased in the rectal mucosa of patients with long-standing ulcerative colitis” Cancer Dec. 1, 1996;78(11):2300-6)

Fragile X syndrome (Hornstra I K et al. “High resolution methylation analysis of the FMR1 gene trinucleotide repeat region in fragile X syndrome” Hum Mol Genet October 1993;2(10):1659-65)

Huntington's disease (Ferluga J et al. “Possible organ and age-related epigenetic factors in Huntington's disease and colorectal carcinoma” Med Hypotheses May 1998;29(1):51-4)

All the above-cited documents are hereby incorporated by reference.

In view of the above, despite the advance in the art of the analysis of gene expression, a screening and/or the diagnosis for a potential disease or medical condition is still a laborious and time consuming task, since in order to achieve a reliable result one has to analyse a vast number of differently expressed genes in parallel.

This makes the analyses unreliable, time consuming, expensive, non-automateable and limits it to the analysis of single genes. No methods exist so far which address these problems to reasonably scale down the effort which has to be applied in order to achieve a result while maintaining its statistical quality.

Therefore, it is an object of the present invention to provide a method which allows for a streamlining of the effort for a reliable cell expression analysis. This analysis shall then be used for less time consuming and more effective diagnostic and therapeutic purposes, in particular in personalised medical treatments.

A further object of the invention is to provide the gene panel which is obtained using a method according to the invention for the use in a treatment of a disease and/or medical condition.

A further object of the invention is to provide systems, methods and computer program products for performing any of the inventive methods.

A further object of the invention is to provide a treatment of a disease and/or medical condition, based on a gene panel according to the invention.

This object is solved according to the present invention by providing a method for the development of gene panels for diagnostic and therapeutic purposes, which comprises the steps of: a)isolating at least one biological sample from each of at least two groups of biological material containing mRNA and/or proteins; b) analysing the expression level of at least one gene in the at least one biological samples; c) selecting the gene(s) exhibiting a different expression level between said at least two groups of biological material, whereby a first knowledge base is generated; d) analysing the level of cytosine methylation in the methylation relevant regions of at least one gene of at least one of the biological samples of step a), wherein the gene is selected on the basis of the first knowledge base; e) selecting the gene(s) exhibiting a different level of cytosine methylation between said at least two groups of biological material, whereby a second knowledge base is generated; and f) adding selected genes from the second knowledge base to a gene panel.

Thus, the present invention provides a method for generating a gene panel combining only the advantages of the presently known expression analysis techniques, which results in a powerful tool for the fast and reliable diagnosis and/or therapy of a enormous number of diseases and/or medical conditions.

Celis et al. (“Gene expression profiling: monitoring transcription and translation products using DNA microarrays and proteomics” FEBS Lett Aug. 25, 2000;480(1):2-16) describe a theoretical approach to combining different technologies such as DNA microarrays and proteomics, which have made possible the analysis of the expression levels of multiple genes simultaneously both in health and disease. In combination, these technologies are said to revolutionise biology, in particular in the area of molecular medicine as they are expected to reveal gene regulation events involved in disease progression as well as to pinpoint potential targets for drug discovery and diagnostics. Celis et al. review the current status of these technologies and highlight some studies in which they have been applied in concert to the analysis of biopsy specimens. Nevertheless, Celis et al. fail to describe or propose the combination of, in particular, data obtained in proteomics expression studies and methylation analyses in order to provide gene panels for further therapeutic or diagnostic purposes.

Other preferred embodiments of the invention will become apparent to the person skilled in the art after reading the features of the dependent claims.

In one embodiment of the method according to the invention, the biological sample is isolated by means of a biopsy, by means of an operation on an individual, by means of a dissection, derived from a preserved biological sample, collected from body fluid(s) and/or collected directly from the environment.

In another embodiment of the method according to the invention, the biological sample comprises a eucaryotic and/or procaryotic cell line, a biopsy sample, blood, sputum, faeces, urine, cerebral liquid, tissue embedded in paraffin, tissue derived from eyes, intestine, brain, heart, prostata, kidney, lung, breast or liver, histological samples or a combination thereof.

A preferred method according to the invention is characterised in that the at least one biological sample is derived from biological material of healthy and/or diseased individuals. Such diseases include all diseases and/or medical conditions which involve a modification of the expression of genes of the cell and include, for example, unwanted side effects of medicaments, cancers, metastasis, dysfunctions, damages or diseases of the central nervous system (CNS), aggressive symptoms or behavioural disorders, clinical, psychological and social consequences of brain injuries, psychotic disorders and disorders of the personality, dementia and/or associates syndromes, cardiovascular diseases, malfunctions or damages, diseases, malfunctions or damages of the gastrointestine, diseases, malfunctions or damages of the respiratory system, injury, inflammation, infection, immunity and/or reconvalescence, diseases, malfunctions or damages as consequences of modifications in the developmental process, diseases, malfunctions or damages of the skin, muscles, connective tissue or bones, endocrine or metabolic diseases, malfunctions or damages, headache, and sexual malfunctions or combinations thereof, leukemia, head and neck cancer, Hodgkin's disease, gastric cancer, prostate cancer, renal cancer, bladder cancer, breast cancer, Burkitt's lymphoma, Wilms tumor, Prader-Willi/Angelman syndrome, ICF syndrome, dermatofibroma, hypertension, pediatric neurobiological diseases, autism, ulcerative colitis, fragile X syndrome, and Huntington's disease.

A preferred method according to the invention is characterised in that the isolation of said biological sample comprises isolating subcellular compartments, organelles, macromolecular structures and multiprotein complexes, partial or complete preparation of the mRNA, reverse transcription or partial digestion of the material with an enzyme selected from proteases, RNAses and/or DNAses or combinations thereof. Such isolations and/preselections can initially even further limit the amount and complexity of the genes which take part in the inventive method.

Further preferred is a method according to the invention in which the analysis of the expression level of the at least one gene in the biological sample comprises determining the relative amount of mRNA or protein derived from said at least one gene. In general, all currently known methods for the expression analysis of genes except methylation analysis can be used for this first step of the inventive method. Preferred are analyses that comprise one or more of one- or two-dimensional gel electrophoresis, differential display, analysis of selected sets of tumour markers, subtractive hybridisation, mass spectrometry, comparative expressed sequence tag sequencing, representational difference analysis, cDNA or oligonucleotide arrays, serial analysis of gene expression, enzymatic, fluorescent, radioactive, dye and/or antibody labelling. Even more preferred are methods according to the invention which comprise measuring intensities of expression during one- or two-dimensional gel electrophoresis, differential display, subtractive hybridisation, DNA, RNA or protein sequencing, mass spectrometry, and enzymatic, radioactive, dye and/or antibody labelling.

In another embodiment of the method according to the invention, the analysis is at least partially performed by means of a suited automate, for example a robot and/or a computer device. Such device would be equipped with the necessary software for the analysis of the expression and could be connected to an inter- or intranet, be part of a neural network or the like. The necessary data/information for the analyses can be present on the system directly or at a remote source, to which the device is directly or indirectly connected, for example via the internet.

In a preferred method according to the invention, the expression levels of at least two genes are analysed in parallel. More preferably, at least 100 genes are analysed in parallel.

In another preferred embodiment of the method according to the invention the selection is based on a combination of the analysis of both mRNA level and protein expression. In another embodiment of the inventive method, the selection is based on the result of at least two individual rows of analyses. This provides for an internal control run of the data which is used for the selection and increases the preciseness of the results. This will further reduce the statistical error for the value of the expression of a selected gene with an only limited increase of the costs for the analysis.

In another preferred method according to the invention, the selection is performed in such a way as to give a first knowledge base comprising only one set of selected genes. Thus, the knowledge base will comprise only “on” and “off” type of data which allows for a very simple decision between expressed or non-expressed genes. In yet another embodiment of the inventive method, the selection is performed in such a way as to give a first knowledge base comprising different subsets of selected genes. Such classes can be referred to as “quality classes” which allows for a much more differentiated selection of the gene panel. The term “quality classes” as used herein comprises all different possibilities of groupings the different expression levels. Such grouping could, for example, include different importance for the selected sites for the analysis of the expression as-well as statistical preciseness and/or quality of the analysis data of the selected gene.

In a preferred method according to the invention, the selection is at least partially performed automatically by means of a suited automate, like a computer device. Such device would be equipped with the necessary software for the analysis of the expression and could be connected to an inter- or intranet, be part of a neural network or the like. The necessary data/information for the analyses can be present on the system directly or at a remote source, to which the device is directly or indirectly connected, for example via the internet.

In a preferred method according to the invention, at least two genes are selected in parallel. More preferably, at least 100 sites are selected in parallel.

According to the invention it is further preferred to analyse methylation relevant regions comprising the complete genes and/or promoters, introns, first exons and/or enhancers of the genes to be analysed. From the analysis of the methylation sites which are relevant for the expression of a certain gene, but not localised inside the sequence of the gene itself, the effect of the site for the expression of the gene can be readily extrapolated by the person skilled in the art.

In another embodiment of the method according to the invention, the analysis of the level of cytosine methylation comprises chemical treatment with bisulphite, hydrogen sulphite or disulphite, polymerase chain reaction (PCR), hybridisation analyses, sequencing, mass spectrometry and fluorescent, enzymatic, radioactive, dye and/or antibody labelling. In general, all methods for the analysis of the methylation statuses at selected sites of the DNA can be employed. Such methods are known to the skilled artisan and are described in, for example, Dahl et al., “Analysis of in vivo methylation.” Methods Mol Biol 2000;130:47-57; Zhou Y. et al., “Use of a single sequencing termination reaction to distinguish between cytosine and 5-methylcytosine in bisulfite-modified DNA.” Biotechniques May 1997;22(5):850-4; Yoder J A et al. “Genetic analysis of genomic methylation patterns in plants and mammals.” C Biol Chem October 1996;377(10):605-10 and others.

Preferred is a method according to the invention in which the analysis is at least partially performed by means of a suited automate, for example a robot. Such device would be equipped with the necessary software for the analysis of the expression and could be connected to an inter- or intranet, be part of a neural network or the like. The necessary data/information for the analyses can be present on the system directly or at a remote source, to which the device is directly or indirectly connected, for example via the internet.

Another preferred method according to the invention is characterised in that all potentially methylated sites of the DNA are analysed. Such sites usually include all so-called “CpG”-islands on a given DNA sequence and are readily detectable by the person skilled in the art. Preferably, the level of cytosine methylation of at least two genes are analysed in parallel. Preferably, the level of at least 100 cytosine methylation sites is analysed in parallel. The analysis of a multitude of sites in parallel allows for both a effective screening and a statistically highly relevant result of the method.

A further preferred method according to the invention is characterised in that the selection is based on the result of at least two individual rows of analyses. This will reduce the statistical error for the value of the methylation sensitivity of a selected site with an only limited increase of the costs for the analysis. In another preferred method according to the invention, the selection is performed in such a way to give a second knowledge base comprising only one set of selected genes. Thus, the knowledge base will comprise only “on” and “off” type of data which allows for a very simple decision between different methylation states.

In yet another embodiment of the inventive method, the selection is performed in such a way to give a second knowledge base comprising different subsets of selected genes. Such classes can be referred to as “quality classes” which allows for a much more differentiated analysis.

In a preferred method according to the invention, the selection is at least partially performed automatically by means of a suited automate, like a computer device. Such device would be equipped with the necessary software for the analysis of the methylation sites and could be connected to an inter- or intranet, be part of a neural network or the like. The necessary data/information for the analyses can be present on the system directly or at a remote source, to which the device is directly or indirectly connected, for example via the internet.

In a preferred method according to the invention, at least two genes are selected in parallel. More preferably, at least 100 genes are selected in parallel.

Another embodiment of the method according to the invention is characterised in that all or a part of the genes of the second knowledge base are added to the gene panel.

In another embodiment of the method according to the invention, additional information about methylation relevant regions of the selected genes is added to the gene panel. This additional information can comprise personal patient data, disease specific data, prior treatment data and/or additional methylation specific data.

Another embodiment of the method according to the invention is characterised in that steps a) to f) are repeated. Repeating the method of the invention suits several different purposes. First, as mentioned above, the statistical quality of the of the resulting data increases. Second, an internal control can be provided, whether the biological sample was taken correctly and resembles e.g. the tissue of interest. The number of repeating “cycles” of the invention can vary depending on the individual case, e.g. depending on the quality of the sample to be analyse. One possibility would be to repeat the method of the invention for at least 5 to 50 times. Preferably, such method according to the invention is characterised in that the method is at least partially performed by means of a suited automate, for example a robot and/or a computer system. The inventive method can be conveniently automated and/or computerized and respective devices and programs are readily known to the person skilled in the art.

In another aspect according to the method according to the invention, the identical biological material, different biological material or a combination thereof is used in step a). Further, in another aspect according to the method according to the invention, the steps are performed in the following order: step a), step d), step e), step b), step c), and step f). This simply interchanges the order of the different steps of the inventive method which should nevertheless lead to a similar or identical result. According to the invention, this method can also at least partially be performed by means of a suited automate, for example a robot.

Another aspect of the present invention is related to a gene panel which is obtained according to a method according to the invention. This gene panel can be further used for the diagnosis and/therapy of different diseases, like, for example, unwanted side effects of medicaments, cancers, metastasis, dysfunctions, damages or diseases of the central nervous system (CNS), aggressive symptoms or behavioural disorders, clinical, psychological and social consequences of brain injuries, psychotic disorders and disorders of the personality, dementia and/or associates syndromes, cardiovascular diseases, malfunctions or damages, diseases, malfunctions or damages of the gastrointestine, diseases, malfunctions or damages of the respiratory system, injury, inflammation, infection, immunity and/or reconvalescence, diseases, malfunctions or damages as consequences of modifications in the developmental process, diseases, malfunctions or damages of the skin, muscles, connective tissue or bones, endocrine or metabolic diseases, malfunctions or damages, headache, and sexual malfunctions or combinations thereof, leukemia, head and neck cancer, Hodgkin's disease, gastric cancer, prostate cancer, renal cancer, bladder cancer, breast cancer, Burkitt's lymphoma, Wilms tumor, Prader-Willi/Angelman syndrome, ICF syndrome, dermatofibroma, hypertension, pediatric neurobiological diseases, autism, ulcerative colitis, fragile X syndrome, and Huntington's disease.

Further preferred is a gene panel according to the invention which comprises additional information data about methylation relevant regions of the selected genes, like the regions of the complete genes and/or promoters, introns, first exons and/or enhancers. According to the invention, the gene panel can be present in the form of a knowledge base on a computer disc, RAM, ROM, or a printed table or listing.

According to the present invention, a “gene panel” designates a knowledge base, listing, table or other information source, that contains information about selected genes, herein also designated as “candidate genes”. According to the present invention, the term “gene panel” should not be understood as merely containing information about the names or designations of the candidate genes. The panel further can contain additional information about the candidate genes, like sequence data, information about the origin (heredity) of the gene, species information, and information about the genetic elements and/or factors that influence expression of the candidate gene(s). Such elements can be the complete genes and/or promoters, introns, first exons and/or enhancers of the candidate genes. Factors can be growth conditions, developmental stage of the biological material from which the candidate gene is derived or other medical data.

In another aspect of the invention, the gene panel can be used for the diagnosis of a disease. Such diseases can include unwanted side effects of medicaments, cancers, metastasis, dysfunctions, damages or diseases of the central nerval system (CNS), aggressive symptoms or behavioural disorders, clinical, psychological and social consequences of brain injuries, psychotic disorders and disorders of the personality, dementia and/or associates syndromes, cardiovascular diseases, malfunctions or damages, diseases, malfunctions or damages of the gastrointestine, diseases, malfunctions or damages of the respiratory system, injury, inflammation, infection, immunity and/or reconvalescence, diseases, malfunctions or damages as consequences of modifications in the developmental process, diseases, malfunctions or damages of the skin, muscles, connective tissue or bones, endocrine or metabolic diseases, malfunctions or damages, headache, and sexual malfunctions or combinations thereof. Particularly preferred is a use according to the invention which is characterised in that the genes are related with leukemia, head and neck cancer, Hodgkin's disease, gastric cancer, prostate cancer, renal cancer, bladder cancer, breast cancer, Burkitt's lymphoma, Wilms tumor, Prader-Willi/Angelman syndrome, ICF syndrome, dermatofibroma, hypertension, pediatric neurobiological diseases, autism, ulcerative colitis, fragile X syndrome, and Huntington's disease.

Another aspect of the present invention is related to a device for the generation of a gene panel according to the invention, which comprises means for generating a first and second knowledge base according to invention and means for adding selected genes from the second knowledge base to a gene panel.

Another aspect of the present invention is related to a method for the diagnosis of a disease, comprising the following steps: a) providing a gene panel according to the invention; b) analysing the level of cytosine methylation at selected sites of the DNA based on said gene panel in biological material of at least one diseased individual with a known disease or medical condition and/or at least one healthy individual, thereby generating a first knowledge base; c) analysing the level of cytosine methylation at selected sites of the DNA based on said gene panel in biological material of at least one diseased individual with an unknown disease or medical condition, thereby generating a second knowledge base; and d) providing a third knowledge base comprising a plurality of expert rules for comparing the first and second knowledge base; e) selecting a type of disease or medical condition for the at least one diseased individual with an unknown disease or medical condition based on said first to third knowledge bases.

A preferred method for the diagnosis of a disease is characterised in that the selected sites of the genes to be analysed are located in the promoters, introns, first exons and/or enhancers or combinations thereof. The inventive method can further comprise the analysis of the level of cytosine methylation comprises chemical treatment with bisulphite, hydrogen sulphite or disulphite, polymerase chain reaction (PCR), hybridisation analyses, sequencing, mass spectrometry and fluorescent, enzymatic, radioactive, dye and/or antibody labelling. Preferably, the method according to the invention is characterised in that the analysis is at least partially performed by means of a suited automate, for example a robot.

Further, in another embodiment of the inventive method, steps b) to d) are repeated before performing step e). Preferably, the identical biological material, different biological material or a combination thereof is used in step c).

In a preferred embodiment, the method according to the invention is at least partially performed by means of a suited automate, for example a robot.

In another aspect of the invention, one embodiment of the inventive method is used for the diagnosis of unwanted side effects of medicaments, cancers, dysfunctions, damages or diseases of the central nerval system (CNS), aggressive symptoms or behavioural disorders, clinical, psychological and social consequences of brain injuries, psychotic disorders and disorders of the personality, dementia and/or associates syndromes, cardiovascular diseases, malfunctions or damages, diseases, malfunctions or damages of the gastrointestine, diseases, malfunctions or damages of the respiratory system, injury, inflammation, infection, immunity and/or reconvalescence, diseases, malfunctions or damages as consequences of modifications in the developmental process, diseases, malfunctions or damages of the skin, muscles, connective tissue or bones, endocrine or metabolic diseases, malfunctions or damages, headache, and sexual malfunctions or combinations thereof. Particularly preferred is a use for the diagnosis of leukemia, head and neck cancer, Hodgkin's disease, gastric cancer, prostate cancer, renal cancer, bladder cancer, breast cancer, Burkitt's lymphoma, Wilms tumor, Prader-Willi/Angelman syndrome, ICF syndrome, dermatofibroma, hypertension, pediatric neurobiological diseases, autism, ulcerative colitis, fragile X syndrome, and Huntington's disease.

In an even further aspect, the invention provides a method for the treatment of a disease or medical condition which comprises a) providing at least one diagnosis according to a method as mentioned above; and b) installing a specific treatment for said at least one diagnosed disease or medical condition. Preferably, said specific treatment is disease specific and/or personalised. In one embodiment this method is used for the treatment of unwanted side effects of medicaments, cancers, dysfunctions, damages or diseases of the central nerval system (CNS), aggressive symptoms or behavioural disorders, clinical, psychological and social consequences of brain injuries, psychotic disorders and disorders of the personality, dementia and/or associates syndromes, cardiovascular diseases, malfunctions or damages, diseases, malfunctions or damages of the gastrointestine, diseases, malfunctions or damages of the respiratory system, injury, inflammation, infection, immunity and/or reconvalescence, diseases, malfunctions or damages as consequences of modifications in the developmental process, diseases, malfunctions or damages of the skin, muscles, connective tissue or bones, endocrine or metabolic diseases, malfunctions or damages, headache, and sexual malfunctions or combinations thereof. Most preferred is a use for the treatment of leukemia, head and neck cancer, Hodgkin's disease, gastric cancer, prostate cancer, renal cancer, bladder cancer, breast cancer, Burkitt's lymphoma, Wilms tumor, Prader-Willi/Angelman syndrome, ICF syndrome, dermatofibroma, hypertension, pediatric neurobiological diseases, autism, ulcerative colitis, fragile X syndrome, and Huntington's disease.

The invention shall now be explained in more detail by the following examples with reference to the attached listing of genes and the accompanying drawings without limiting the scope of the concept of the invention.

The attached sequence listing shows a panel of genes, whose methylation statuses are altered depending on cellular states as indicated above each subgroup of the listing.

### EXAMPLES

### Example 1

Proteomics Plus Subsequent Methylation Screening

In this example, a proteomics-derived step was used in order to analyse the expression level of a set of proteins. First, a 2-D Gelelectrophoresis according to standard protocols (see above) was perfomed for both a prostate cancer cell line and cells derived from a healthy prostate in which a staining with Sypro Ruby dye was used. Then, the resulting gels were scanned using a CCD-camera and the scanned picture were analysed using a computer-based analysis software, e.g. “Imagemaster” (Amersham-Pharmacia) or “Z3” (Compugen).

Proteins that were differently expressed in both analysis pattern were excised by a robot (Flexys robot, genomic solutions) and tryptically digested. The peptides were analysed using a MALDI-TOF mass spectrometry. The resulting fragments were analysed via peptide mapping and compared to the protein database (which one). Finally, the differently expressed proteins were listed and displayed. This listing included several differently expressed proteins.

On the basis of the listing obtained in the first step, a methylation analysis was performed according to the methods described above. The analysis revealed, that the methylation statuses of the several proteins were significantly different compared to the non-prostate cancer cell line.

Taken together, these selected genes can be further used in diagnosis and/or therapy as a minimal set of markers for prostate cancer while providing a result of maximal reliably.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

1. A method for the development of gene panels for diagnostic and therapeutic purposes, comprising the steps of:
a)isolating at least one biological sample from each of at least two groups of biological material containing mRNA and/or proteins;
b)analysing the expression level of at least one gene in at least one of the biological samples;
c)selecting the gene(s) exhibiting a different expression level between said at least two groups of biological material,
whereby a first knowledge base is generated;
d)analysing the level of cytosine methylation in the methylation relevant regions of at least one gene of at least one of the biological samples of step a), wherein the gene is selected on the basis of the first knowledge base;
e)selecting the gene(s) exhibiting a different level of cytosine methylation between said at least two groups of biological material,
whereby a second knowledge base is generated; and
f)adding selected genes from the second knowledge base to a gene panel.

2. Method according to claim 1, comprising that the biological material is isolated by means of a biopsy, by means of an operation on an individual, by means of a dissection, derived from a preserved biological sample, collected from body fluid(s) and/or collected directly from the environment.

3. Method according to claim 1 or 2, characterised in that the biological material comprises a eucaryotic and/or procaryotic cell line, a biopsy sample, blood, sputum, faeces, urine, cerebral liquid, tissue embedded in paraffin, tissue derived from eyes, intestine, brain, heart, prostata, kidney, lung, breast or liver, histological samples or a combination thereof.

4. Method according to any of claims 1 to 3, characterised in that at least one of the biological samples is derived from biological material of healthy and/or diseased individuals.

5. Method according to any of claims 1 to 4, characterised in that the isolation of said biological sample comprises isolating subcellular compartments, organelles, macromolecular structures and multiprotein complexes, partial or complete preparation of the mRNA, reverse transcription or partial digestion of the material with an enzyme selected from proteases, RNAses and/or DNAses or combinations thereof.

6. Method according to any of claims 1 to 5, characterised in that the analysis of the expression level of the at least one gene in the biological sample comprises determining the relative amount of mRNA or protein derived from said at least one gene.

7. Method according to claim 6, characterised in that the analysis comprises one- or two-dimensional gel electrophoresis, differential display, analysis of selected sets of tumour markers, subtractive hybridisation, mass spectrometry, comparative expressed sequence tag sequencing, representational difference analysis, cDNA or oligonucleotide arrays, serial analysis of gene expression, enzymatic, fluorescent, radioactive, dye and/or antibody labelling.

8. Method according to claim 7, characterised in that the analysis further comprises measuring intensities of expression during one- or two-dimensional gel electrophoresis, differential display, subtractive hybridisation, DNA, RNA or protein sequencing, mass spectrometry, and enzymatic, radioactive, dye and/or antibody labelling.

9. Method according to any of claims 6 to 8, characterised in that the analysis is at least partially performed by means of a suited automate, for example a robot.

10. Method according to any of claims 6 to 9, characterised in that the expression levels of at least two genes are analysed in parallel.

11. Method according claim 10, characterised in that the expression levels of at least 100 genes are analysed in parallel.

12. Method according to any of claims 1 to 11, characterised in that the selection is based on a combination of the analysis of both mRNA level and protein expression.

13. Method according to any of claims 1 to 12, characterised in that the selection is based on the result of at least two individual rows of analyses.

14. Method according to any of claims 1 to 13, characterised in that the selection is performed in such a way as to give a first knowledge base comprising only one set of selected genes.

15. Method according to any of claims 1 to 13, characterised in that the selection is performed in such a way as to give a first knowledge base comprising different subsets of selected genes.

16. Method according to any of claims 1 to 13, characterised in that the selection is at least partially performed automatically by means of a suited automate, such as a computer device.

17. Method according to any of claims 1 to 16, characterised in that at least two genes are selected in parallel.

18. Method according to claim 17, characterised in that at least 100 genes are selected in parallel.

19. Method according to any of claims 1 to 18, characterised in that the methylation relevant regions comprise the complete genes and/or promoters, introns, first exons and/or enhancers of the genes to be analysed.

20. Method according to any of claims 1 to 19, characterised in that the analysis of the level of cytosine methylation comprises chemical treatment with bisulphite, hydrogen sulphite or disulphite, polymerase chain reaction (PCR), hybridisation analyses, sequencing, mass spectrometry and fluorescent, enzymatic, radioactive, dye and/or antibody labelling.

21. Method according to any of claims 1 to 20, characterised in that the analysis is at least partially performed by means of a suited automate, for example a robot.

22. Method according to any of claims 1 to 21, characterised in that the level of cytosine methylation of at least two genes are analysed in parallel.

23. Method according claim 22, characterised in that the level of cytosine methylation of at least 100 genes are analysed in parallel.

24. Method according to any of claims 1 to 23, characterised in that the selection is based on the result of at least two individual rows of analyses.

25. Method according to any of claims 1 to 24, characterised in that the selection is performed in such a way as to give a second knowledge base comprising only one set of selected genes.

26. Method according to any of claims 1 to 25, characterised in that the selection is performed in such a way as to give a second knowledge base comprising different subsets of selected genes.

27. Method according to any of claims 1 to 26, characterised in that the selection is a t l east partially performed automatically by means of a suited automate, such as a computer device.

28. Method according to any of claims 1 to 27, characterised in that at least two genes are selected in parallel.

29. Method according to claim 28, characterised in that at least 100 genes are selected in parallel.

30. Method according to any of claims 1 to 29, characterised in that all or a part of the genes of the second knowledge base are added to the gene panel.

31. Method according to any of claims 1 to 30, characterised in that additional information about methylation relevant regions of the selected genes is added to the gene panel.

32. Method according to any of claims 1 to 31, characterised in that steps a) to f) are repeated.

33. Method according to claim 32, characterised in that it is repeated for at least 100 times.

34. Method according to any of claims 32 or 33,characterised in that the identical biological material, different biological material or a combination thereof is used in step a).

35. Method according to any of claims 1 to 34, characterised in that the steps are performed in the following order: step a), step d), step e), step b), step c), and step f).

36. Method according to any of claims 1 to 35, characterised in that it is at least partially performed by means of a suited automate, for example a robot.

37. Gene panel, obtainable according to a method according to any of claims 1 to 36.

38. Gene panel according to claim 37 comprising additional information data about methylation relevant regions of the selected genes, like the complete genes and/or promoters, introns, first exons and/or enhancers of the selected genes.

39. Gene panel according to claim 37 or 38 in the form of a knowledge base on a computer disc, RAM, ROM, or a printed table or listing.

40. Use of a gene panel according to any of claims 37 to 39 for the diagnosis of a disease.

41. Device for the generation of a gene panel according to any of claims 37 to 39, comprising
means for generating a first and second knowledge base according to claim 1; and
means for adding selected genes from the second knowledge base to a gene panel.

42. A method for the diagnosis of a disease, comprising the following steps:
a) providing a gene panel according to any of claims 37 to 39;
b) analysing the level of cytosine methylation at selected sites of the DNA based on said gene panel in biological material of at least one diseased individual with a known disease or medical condition and/or at least one healthy individual, thereby generating a first knowledge base;
c) analysing the level of cytosine methylation at selected sites of the DNA based on said gene panel in biological material of at least one diseased individual with an unknown disease or medical condition, thereby generating a second knowledge base; and
d) providing a third knowledge base comprising a plurality of expert rules for comparing the first and second knowledge base;
e) selecting a type of disease or medical condition for the at least one diseased individual with an unknown disease or medical condition bas ed on said first to third knowledge bases.

44. Method according to claim 43, characterised in that the selected sites of the genes to be analysed are located in the promoters, introns, first exons and/or enhancers or combinations thereof.

45. Method according to claims 43 or 44, characterised in that the analysis of the level of cytosine methylation comprises chemical treatment with bisulphite, hydrogen sulphite or disulphite, polymerase chain reaction (PCR), hybridisation analyses, sequencing, mass spectrometry and fluorescent, enzymatic, radioactive, dye and/or antibody labelling.

46. Method according to any of claims 43 to 45, characterised in that the analysis is at least partially performed by means of a suited automate, for example a robot.

47. Method according to any of claims 43 to 46, characterised in that the level of cytosine methylation of at least two genes are analysed in parallel.

48. Method according claim 47, characterised in that the level of cytosine methylation of at least 100 genes are analysed in parallel.

49. Method according to any of claims 43 to 48, characterised in that steps b) to d) are repeated before performing step e).

50. Method according to claim 43 to 49, characterised in that it is repeated for at least 100 times.

51. Method according to any of claims 43 to 50, characterised in that the identical biological material, different biological material or a combination thereof is used in step c).

52. Method according to any of claims 43 to 51, characterised in that it is at least partially performed by means of a suited automate, for example a robot.

53. Use of a method according to any of claims 43 to 52 for the diagnosis of unwanted side effects of medicaments, cancers, dysfunctions, damages or diseases of the central nerval system (CNS), aggressive symptoms or behavioural disorders, clinical, psychological and social consequences of brain injuries, psychotic disorders and disorders of the personality, dementia and/or associates syndromes, cardiovascular diseases, malfunctions or damages, diseases, malfunctions or damages of the gastrointestine, diseases, malfunctions or damages of the respiratory system, injury, inflammation, infection, immunity and/or reconvalescence, diseases, malfunctions or damages as consequences of modifications in the developmental process, diseases, malfunctions or damages of the skin, muscles, connective tissue or bones, endocrine or metabolic diseases, malfunctions or damages, headache, and sexual malfunctions or combinations thereof.

54. Use according to claim 53 for the diagnosis of leukemia, head and neck cancer, Hodgkin's disease, gastric cancer, prostate cancer, renal cancer, bladder cancer, breast cancer, Burkitt's lymphoma, Wilms tumor, Prader-Willi/Angelman syndrome, ICF syndrome, dermatofibroma, hypertension, pediatric neurobiological diseases, autism, ulcerative colitis, fragile X syndrome, and Huntington's disease.

55. Method for the treatment of a disease or medical condition, comprising
a) providing at least one diagnosis according to a method according to any of claims 43 to 52; and
b) installing a specific treatment for said at least one diagnosed disease or medical condition.

56. Method according to claim 55, wherein said specific treatment is disease specific and/or personalised.

57. Use of a method according to claims 55 or 56 or for the treatment of unwanted side effects of medicaments, cancers, dysfunctions, damages or diseases of the central nerval system (CNS), aggressive symptoms or behavioural disorders, clinical, psychological and social consequences of brain injuries, psychotic disorders and disorders of the personality, dementia and/or associates syndromes, cardiovascular diseases, malfunctions or damages, diseases, malfunctions or damages of the gastrointestine, diseases, malfunctions or damages of the respiratory system, injury, inflammation, infection, immunity and/or reconvalescence, diseases, malfunctions or damages as consequences of modifications in the developmental process, diseases, malfunctions or damages of the skin, muscles, connective tissue or bones, endocrine or metabolic diseases, malfunctions or damages, headache, and sexual malfunctions or combinations thereof.

58. Use according to claim 57 for the treatment of leukemia, head and neck cancer, Hodgkin's disease, gastric cancer, prostate cancer, renal cancer, bladder cancer, breast cancer, Burkitt's lymphoma, Wilms tumor, Prader-Willi/Angelman syndrome, ICF syndrome, dermatofibroma, hypertension, pediatric neurobiological diseases, autism, ulcerative colitis, fragile X syndrome, and Huntington's disease.
